(19) United States
(12) Patent Application Publication
(10) Pub. No.: US 2016/0000858 A1
(43) Pub. Date: Jan. 7, 2016

(54) COMPOSITIONS AND METHODS FOR MITIGATING SKIN IRRITATION AND ENHANCING SKIN BARRIER FUNCTION

(71) Applicant: GOJO INDUSTRIES, INC., Akron, OH (US)

(72) Inventors: Jessica R. Tittl, Chagrin Falls, OH (US); Christopher A. Wolf, Ashland, OH (US)

(21) Appl. No.: 14/750,241

(22) Filed: Jun. 25, 2015

Related U.S. Application Data
(60) Provisional application No. 62/021,310, filed on Jul. 7, 2014.
(60) Provisional application No. 62/077,349, filed on Nov. 10, 2014.

(51) Int. Cl. A61K 38/08; A61K 8/64; A61Q 19/00; A61K 9/00

(52) U.S. Cl. CPC A61K 38/08; A61K 9/0014; A61K 8/645; A61Q 19/00; A61K 2800/74

(57) ABSTRACT

Methods and compositions for cell regeneration and decreased irritation of mammalian skin cells are provided, wherein the compositions include a synergistic combination of two or more oligopeptides. The compositions may advantageously be applied to mammalian skin to effect a regeneration of skin cells and to effect a reduction in inflammation and redness experienced by the skin. Skin cleansing and sanitizing compositions are provided that include at least one oligopeptide, wherein the compositions have reduced irritancy potential.

### TECHNICAL FIELD

One or more embodiments of the present invention provide methods and compositions for mitigating the irritation of mammalian skin cells, protecting cell viability and/or enhancing cell-cell junction, thus improving skin barrier function. More particularly, it relates to compositions containing at least one or more oligopeptide and an acceptable carrier. The compositions can be applied to mammalian skin to reduce the inflammation and redness that can result from the use of skin irritants.

### BACKGROUND OF THE INVENTION

Many ingredients in skin care and cosmetic products can cause skin irritation. Surfactants such as sodium lauryl sulfate (SLS) are known to be skin irritants. Retinoid and its derivatives, can cause severe local irritation manifested as mild erythema and stratum corneum peeling of the skin. Topical or systemic use of some skin cleansers and disinfectants is linked to skin irritation.

Ingredients such as benzyol peroxide, alpha-hydroxyl acids and derivatives thereof, salicylic acid, natural plant extracts, sunscreen actives, urea, and preservatives are also known to cause external skin irritations. Furthermore, skin irritations may be caused by inherent disease conditions such as acne, rosacea, atopic dermatitis, and other disease states. Typical approaches to reduce irritation include reducing the concentration of the inflammatory ingredient, use of alternatives or formulation/delivery approaches, such as encapsulation, controlled release, compartmentalization, inclusion of non-irritating excipients. None of the above has successfully reduced irritation while retaining efficacy. As a result, there is a need for anti-irritant substances to mitigate external skin irritations, or irritations caused by inherent skin conditions.

Skin exposure to water and typical cleansers may have a negative effect on the stratum corneum (SC) structure and function. Effects include disruption of the lipid bilayer architecture to create defects or holes in the barrier. As a result, the barrier becomes more permeable, allowing irritants and microorganisms to penetrate into and through the uppermost layers of the skin. In cases of severe hand irritation, cracks or fissures (with or without bleeding) may develop indicating damage to the dermis. The skin's response to these damaging effects is immediate, but the accelerated efforts to repair the barrier and generate new stratum corneum leads to imperfect architecture, when compared to stratum corneum that is formed during the normal course of SC replacement. The rapidly-produced SC has poor water binding properties, leading to insufficient skin moisture and inadequate desquamation.

Under normal conditions, there is also a constant loss of SC cells, as individual units from the surface of the skin, and new cells move from the bottom of the SC to the surface, generally over a period of about 14 days. When skin moisture is too low, the SC cells come off of the skin surface as clumps of cells, observed as dry scales. There are ingredients in most skin care and cosmetic products that accelerate the loss of SC cells, by affecting cell viability and/or by modulating epidermal proliferation. There is a substantial need for products that protect cell viability and proliferation, while at the same time decreasing irritation of the skin cells caused by exposure to water and typical cleansers.

Acetyl hexapeptides such as acetyl hexapeptide-3 have been employed for properties including anti-wrinkle, collagen boosting, anti-aging, and relaxing of facial tension. Acetyl hexapeptide-3 is said to be non-irritating. Some peptides have been described as capable of stimulating collagen synthesis, and increasing the hydration of the skin.

However, acetyl hexapeptides have not heretofore been described as mitigating the skin irritation of surfactants, nor have they been described as protecting cell viability against ingredients found in topical hand sanitizers, washes and lotions.

### BRIEF DESCRIPTION OF THE DRAWINGS

FIG. 1 is a graphical representation of the effect of compositions containing oligopeptides on the irritation response of cells treated with known irritants, as quantified by measuring IL-8 secretion.

FIG. 2 is a graphical representation of the reduction of IL-8 secretion for test samples, compared to Control B, which contained no oligopeptide.

FIG. 3 is a graphical representation of the effect of oligopeptides on cell binding, as measured by Real-Time Quantitative Reverse Transcription Polymerase Chain Reaction (qRT-PCR) for desmoglein (DSG1).

FIG. 4 is a graphical representation of the effect of oligopeptides on cell binding, as measured by qRT-PCR for democollin (DSC3).

### DETAILED DESCRIPTION OF ILLUSTRATIVE EMBODIMENTS

In one or more embodiments, compositions of the present invention include at least one oligopeptide, one or more cosmetically or pharmaceutically acceptable carriers, and optionally one or more benefit agents.

Generally, oligopeptides are short chains of amino acid moieties linked by amide bonds. Any combination of amino acids may be included.

In one or more embodiments, the oligopeptide includes 12 amino acid moieties or less, in other embodiments, 10 amino acid moieties or less, and in other embodiments, 8 amino acid moieties or less.

In one or more embodiments, the peptide includes at least 5 amino acid moieties. In one or more embodiments, the peptide includes at least 6 amino acid moieties. In one or more embodiments, the peptide includes from 5 to 6 amino acid moieties. Longer peptides are believed to have at least the potential for causing undesirable responses on application to the human body. In one or more embodiments, the stereochemistry of the amino acids of the peptide is L—.

In one or more embodiments, the oligopeptide may be identified as a sequence of amino acids, such as Ser.Pro.Ala.Gly.Gly.Pro. In other embodiments, the oligopeptide may be identified as a chemical structure, such as:

R₁—Wₙ—Xₘ-AA₁-AA₂-AA₃-AA₄-AA₅-AA₆-Yₚ—Z_{q}—R₂ (I)

or may include stereoisomers, mixtures thereof and/or cosmetically or pharmaceutically acceptable salts thereof, wherein:
AA₁ is selected from the group formed by -Ser-, -Thr- and -Tyr-;
AA₂ is selected from the group formed by -Pro- and -Val-;
AA₃ is selected from the group formed by -Ala- and -Gly-;
AA₄ is selected from the group formed by -Glu-, -Gly- and -Val-;
AA₅ is selected from the group formed by -Gly- and -Ala-;
AA₆ is selected from the group formed by -Gln-, -Gly-, -His- and -Pro-;
W, X, Y, Z are amino acids and are independently selected from amongst themselves;
n, m, p and q are independently selected from amongst themselves and have a value of 0 or 1;
n+m+p+q is lower or equal to 2;
R₁ is selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl and R₅—CO—, wherein R₅ is selected from the group formed by H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl;
R₂ is selected from the group formed by —NR₃R₄, —OR₃ and —SR₃, wherein R₃ and R₄ are independently selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and with the condition that R₁ and R₂ are not α-amino acids;
when AA₃ is -Gly- and AA₆ is -Pro-, then AA₂ is -Val-; and
when W or X are -Val-, Y or Z are -Arg-, AA₁ is -Ser-, AA₂ is -Pro-, AA₄ is -Glu-, AA₅ is -Ala- and AA₆ is -Gln-, then AA₃ is -Gly-.

Groups R1 and R2 are respectively bound to the amino-terminal (N-terminal) and carboxy-terminal (C-terminal) of the peptide sequences.

In other embodiments, the oligopeptide may be identified by an INCI (International Nomenclature of Cosmetic Ingredients) name, such as acetyl hexapeptide followed by a numerical designation. Oligopeptides are further described in U.S. Published Patent Application No. 2014/0120141 A1, which is hereby incorporated by reference.

In one or more embodiments, compositions of the present invention include at least one acetyl hexapeptide.

Generally, hexapeptides contain six amino acid moieties. Some non-limiting examples of acetyl hexapeptides are acetyl hexapeptide-1, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, and acetyl hexapeptide-46, acetyl hexapeptide-49.

Acetyl Hexapeptide-1 is reaction product of alanine, arginine, histidine, leucine, phenylalanine and tryptophane hexapeptide with acetic acid. Acetyl hexapeptide-1 is commercially available, for example from Lucas Meyer Cosmetics as a blend with glycerin and water and dextran under the tradename Melitane®.

Acetyl hexapeptide-8 is commercially available, for example from Theraderm Clinical Skin Care under the tradename Argireline®.

Acetyl hexapeptide-30 is commercially available, for example from Lipotec LLC under the tradename Inyline®.

Acetyl hexapeptide-38 is commercially available, for example from Lipotec LLC as a blend with butylene glycol and water, under the tradename Adifyline™

Acetyl hexapeptide-39 is commercially available, for example from Lipotec LLC under the tradename Silusyne®.

Acetyl hexapeptide-46 is commercially available, for example from Lipotec LLC as a blend with butylene glycol, water and citric acid, under the tradename Delisens®.

Other acetyl hexapeptides are also commercially available.

In one or more embodiments, the oligopeptide is selected from the group consisting of acetyl hexapeptide-38 and acetyl hexapeptide-46.

In certain embodiments, a synergistic effect is seen when two or more oligopeptides are combined. In other words, the reduction in irritancy when two or more oligopeptides are combined is greater than just an additive effect. In one or more embodiments, the positive effect on cell regeneration is more than the sum of the improvement that is seen with equivalent amounts of the individual components.

Thus, in one or more embodiments, compositions of the present invention comprise two or more oligopeptides. In one or more embodiments, compositions of the present invention comprise two or more acetyl hexapeptides. In one or more embodiments, compositions of the present invention comprise two or more acetyl hexapeptides, and at least one of the acetyl hexapeptides is selected from acetyl hexapeptide-38 and acetyl hexapeptide-46. In one or more embodiments, compositions of the present invention comprise acetyl hexapeptide-38 and acetyl hexapeptide-46.

In one or more embodiments, compositions of the present invention include at least one pentapeptide. Generally, pentapeptides contain five amino acid moieties. Some non-limiting examples of pentapeptides include acetyl pentapeptide-1, pamitoyl pentapeptide-3, pamitoyl pentapeptide-4, and myristoyl pentapeptide-17. In one or more embodiments, compositions of the present invention comprise pamitoyl pentapeptide-3. In one or more embodiments, compositions of the present invention comprise pamitoyl pentapeptide-4. In one or more embodiments, compositions of the present invention comprise myristoyl pentapeptide-17.

Acetyl pentapeptide-1 is commercially available, for example from Spec Chem Ind. Under the tradename SpecPed SC-AP1. Pamitoyl pentapeptide-3 is commercially available, for example from Spec Chem Ind. Under the tradename SpecPed SC-PP3. Myristoyl pentapeptide-17 is commercially available, for example from Spec Chem Ind. Under the tradename SpecPed SC-MP 17.

Another aspect of the invention is a cosmetic or pharmaceutical composition which comprises at least one oligopeptide described above together with at least one cosmetically or pharmaceutically acceptable adjuvant. The oligopeptides can be incorporated into cosmetic or pharmaceutical delivery and/or sustained release systems. These compositions can be prepared by conventional means known to persons skilled in the art [“Harry's Cosmeticology”, Seventh edition, (1982), Wilkinson J. B., Moore R. J., ed. Longman House, Essex, GB].

In one or more embodiments, the term “delivery systems” relates to a diluent, adjuvant, excipient or carrier with which the peptide of the invention is administered. In one or more embodiments, these cosmetic or pharmaceutical carriers can be liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin and similar.

In one or more embodiments, the term “sustained release” relates to a delivery system of a compound which provides the gradual release of this compound during a period of time and preferably, although not necessarily, with relatively constant compound release levels over a period of time.

The compositions of topical or transdermal application may be produced in any solid, liquid or semisolid formulation, such as and not restricted to, creams, multiple emulsions such as and not restricted to, oil and/or silicone in water emulsions, water-in-oil and/or silicone emulsions, water/oil/water or water/silicone/water type emulsions, and oil/water/oil or silicone/water/silicone type emulsions, anhydrous compositions, aqueous dispersions, oils, milks, balsams, foams, lotions, gels, cream gels, hydroalcoholic solutions, hydroglycolic solutions, hydrogels, liniments, sera, soaps, shampoos, conditioners, serums, polysaccharide films, ointments, mousses, pomades, powders, bars, pencils and sprays or aerosols, including leave-on and rinse-off formulations.

Advantageously, the acetyl hexapeptides can be employed in a wide variety of topical products in order to mitigate the irritancy of the product, or to enhance the skin-conditioning benefits of the product. Topical products in which the acetyl hexapeptide can be employed include aqueous or alcohol-based sanitizers, surfactant-based washes, lotions, serums, shampoos and conditioners, creams, aqueous based leave on gels, sprayable sanitizers, sprayable lotions, sunscreens, aftersun care, eye creams, lip products.

The compositions of this invention are preferably in the form of topical products that can be applied externally to the skin and can be prepared in accordance with conventional techniques known to those of ordinary skill in the art. The delivery system may take a variety of physical forms such as, for example, creams, dressings, gels, lotions, ointments or liquids. In one or more embodiments, compositions of the present invention may be formulated as a foamable composition, a thickened gel composition, a sprayable liquid, a rinse, or may be applied to a wipe.

Suitable delivery systems include aqueous or alcohol-based sanitizer compositions, such as those described in U.S. Pat. Nos. 4,956,170, 6,143,710, 6,183,766, 6,228,385, 6,248,343, 7,803,390, 8,293,802, 8,323,633, 8,329,758, 8,338,491, 8,697,103, all of which are hereby incorporated by reference.

Suitable delivery systems also include surfactant-based sanitizer compositions, such as those described in U.S. Pat. Nos. 5,712,232, 5,972,860, 6,479,442, 6,413,921, 7,517,842, and 8,372,790, all of which are hereby incorporated by reference.

Suitable carriers also include lotions. Examples of lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

These same general ingredients may be formulated into a cream rather than a lotion, or into gels, or into solid sticks by utilization of different proportions of the ingredients and/or by inclusion of thickening agents such as gums or other forms of hydrophilic colloids.

The cosmetically or pharmaceutically effective amount of the peptides of the invention which should be administered, as well as their dosage, will depend on numerous factors, including age, state of the patient, the nature or severity of the condition, disorder or disease to be treated and/or cared for, the route and frequency of administration and of the particular nature of the peptides to be used.

In one or more embodiments, compositions of the present invention comprise at least an effective amount of the oliopeptide, wherein an effective amount is the amount that mitigates skin irritation or enhances skin conditioning, when compared to the same composition but not containing any oligopeptide. In one or more embodiments, an effective amount of oligopeptide is at least about 0.06 parts per million by weight (ppm), based upon the total weight of the composition. In other embodiments, an effective amount is at least about 0.10 ppm, in other embodiments at least about 0.12 ppm, based upon the total weight of the composition.

In one or more embodiments, the composition comprises from about 0.06 to about 100 ppm of oligopeptide, based upon the total weight of the composition. In one or more embodiments, the composition comprises from about 0.08 to about 50 ppm of oligopeptide, based upon the total weight of the composition. In one or more embodiments, the composition comprises from about 0.1 to about 30 ppm of oligopeptide, based upon the total weight of the composition. In one or more embodiments, the composition comprises from about 0.5 to about 25 ppm of oligopeptide, based upon the total weight of the composition.

The oligopeptides of this invention may have variable solubility in water. Water-soluble oligopeptides may be incorporated directly into aqueous compositions. Water-insoluble oligopeptides and those with limited water solubility may be solubilized in cosmetically or pharmaceutically acceptable solvents such as and not restricted to, ethanol, propanol, isopropanol, propylene glycol, glycerin, butylene glycol or polyethylene glycol or any combination thereof.

In one or more embodiments, the oligopeptide may be added to the composition as a solution or emulsion. In other words, the oligopeptide may be premixed with a carrier, and optionally one or more other ingredients, to form an oligopeptide solution or emulsion, with the proviso that the carrier does not deleteriously affect the beneficial properties of the composition.

Examples of carriers include water, alcohol, or blends of water and another carrier such as glycols, ketones, linear and/or cyclic hydrocarbons, triglycerides, carbonates, silicones, alkenes, esters such as acetates, benzoates, fatty esters, glyceryl esters, ethers, amides, polyethylene glycols, PEG/PPG copolymers, inorganic salt solutions such as saline, and mixtures thereof. It will be understood that, when the oligopeptide is premixed to form an oligopeptide solution or emulsion, the amount of solution or emulsion that is added to the composition is selected so that the amount of oligopeptide falls within the ranges set forth hereinabove.

Compositions or the present invention may further comprise one or more of a wide range of optional ingredients, with the proviso that they do not deleteriously affect the beneficial properties of the composition. The CTFA International Cosmetic Ingredient Dictionary and Handbook, Twelfth Edition 2008, and the 2007 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Examples of optional ingredients, classified by function, include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, humectants, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, and keratolytics, topical active ingredients, and the like. contains one or more optional benefit agents selected from the group consisting of primary skin-conditioning agents, deposition enhancers, humectants, moisturizing esters, emulsifying agents, silicone glycols, miscellaneous skin conditioners, thickeners, and/or antimicrobial agents.

In one or more embodiments, strong acids and other ingredients that may attack the peptide bonds in the oligopeptide may be limited. In one or more embodiments, the amount of protein denaturants is limited. In one or more embodiments, elevated temperatures are avoided.

The composition may be prepared by simply mixing the components together. The order of addition is not particularly limited, but may advantageously be selected based upon the solubility of the various ingredients.

In one or more embodiments, the composition is topically applied to skin. In one or more embodiments, the composition may be topically applied to an affected skin area in a predetermined or as-needed regimen. In one or more embodiments, the composition is included as part of a skin cleansing or sanitizing regimen.

Advantageously, in one or more embodiments, skin cleansers and sanitizers containing one or more oligopeptide according to the present invention have a reduced irritancy potential when compared to the same skin cleanser or sanitizer but not containing one or more oligopeptide according to the present invention.

Thus, the present invention further provides a method for reducing the irritancy potential of a skin cleanser or sanitizer. The method includes the step of combining a skin cleanser or sanitizer composition with one or more oligopeptides prior to form a less irritating skin cleanser or sanitizer composition. The method includes the further step of contacting the skin with the less irritating composition for a period sufficient to cleanse and/or sanitize the skin. In one or more embodiments, when the amount of skin irritation is measured, as for example by testing the IL-8 secretion, the amount of skin irritation is reduced, compared to when the method is repeated but using the same skin cleanser or sanitizer composition without any oligopeptide. In one or more embodiments, compositions containing two or more oligopeptides provide a synergistic reduction of the skin irritation potential of the compositions.

Advantageously, in one or more embodiments, skin cleansers and sanitizers containing one or more oligopeptide according to the present invention enhance the skin barrier function, when compared to the same skin cleanser or sanitizer but not containing one or more oligopeptide according to the present invention.

Thus, the present invention further provides a method for improving the effect of a skin cleanser or sanitizer on skin barrier function. The method includes the step of combining a skin cleanser or sanitizer composition with one or more oligopeptides prior to form an enhanced skin cleanser or sanitizer composition. The method includes the further step of contacting the skin with the enhanced composition for a period sufficient to cleanse and/or sanitize the skin. In one or more embodiments, when the skin barrier function is assessed, as for example by testing the skin for cell adhesion proteins, the amount of skin barrier function is improved, compared to when the method is repeated but using the same skin cleanser or sanitizer composition without any oligopeptide. In one or more embodiments, compositions containing two or more oligopeptides provide a synergistic enhancement of the skin barrier function.

Advantageously, compositions and methods of the present invention may be useful to treat a variety of skin conditions that result in inflammation or erythema. For example, inflammation or erythema can result from external causes such as sun or wind burn or irritating soaps or cleansers. It is also known that inflammation and erythema can be caused from inherent conditions such as rosacea, atopic dermatitis, or allergic skin reactions.

In order to demonstrate the practice of the present invention, the following examples have been prepared and tested. The examples should not, however, be viewed as limiting the scope of the invention. The claims will serve to define the invention.

### EXAMPLES

### Testing Methods

IL-*ELISA

Interleukin 8 (IL-8) is a chemokine and proinflammatory cytokine produced by macrophages and other cell types such as epithelial cells. It is secreted from keratinocytes in skin in response to inflammatory stimuli. IL-8 is secreted and is an important mediator of the immune reaction in the innate immune system response. IL-8 overexpressed is a biomarker of skin irritation.

For Control A, human dermal keratinocytes are left untreated. No irritation is expected, and therefore Control A provides a baseline. For Control B, IL-8 is induced in human dermal keratinocytes by applying a surfactant mixture that is a combination of sodium laureth sulfate and polyquaternium-10. For all other samples, the human dermal keratinocytes are co-treated with the surfactant mixture and a composition containing the ingredient of interest. Decreased 11-8 expression reflects the ingredient's anti-irritation activity.

In order to carry out the test method, an assay kit was employed that was obtained from R&D Systems: Human CXCL8/IL-8 Quantikine ELISA Kit.

The following steps were followed: 1. Bring all reagents and samples to room temperature before use. 2. Prepare all reagents, standard dilutions, and samples. 3. Remove excess microplate strips from the plate frame, return them to the foil pouch containing the desiccant pack, and reseal. 4. Add 100 μL of Assay Diluent to each well. 5. Add 50 μL of

Standard, control, or sample to each well. Cover with a plate sealer, and incubate at room temperature for 2 hours. 6. Aspirate each well and wash, repeating the process 3 times for a total of 4 washes. 7. Add 100 μL of Conjugate to each well. Cover with a new plate sealer, and incubate at room temperature for 1 hour. 8. Aspirate and wash 4 times. 9. Add 200 μL Substrate Solution to each well. Incubate at room temperature for 30 minutes, making sure to protect the wells from the light. 10. Add 50 μL of Stop Solution to each well. The liquid was removed from the well and, using a colorimeter, absorbance was measured at 450 nanometers (nm) within 30 minutes. Wavelength correction was set to 540 nm or 570 nm.

MTT Assay

The MTT assay is a colorimetric assay for assessing cell viability, cell proliferation, and/or cytotoxicity. NAD(P)H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to insoluble formazan, which has a purple color. MTT assay can also be used to measure cytotoxicity (loss of viable cells) or cytostatic activity (shift from proliferative to resting status) of potential medicinal agents and toxic materials.

Controls A and B described above for the IL-8 Assay were also employed in this test. The mitigating effect of the test samples on the effect of Control B on the keratinocytes was measured. More specifically, while Control B has a negative effect on cell viability, cell proliferation, and/or cytotoxicity, this mitigation of this negative effect was determined by measuring the reduction of MTT.

The following steps were followed; Once the liquid was removed from the wells for the IL-8 Assay described above, 100 μl/well of 0.5 mg/ml of MTT in phenol red-free DMEM (cell culture medium) was added into each of the 96-well plates. After incubating 1 hour at 37° C., all liquid was removed (MTT solution) from the wells of the culture plate. Then 100 μl of DMSO was added to each well to completely dissolve the purple product. Absorption was measured using a plate reader at 550 nm wavelength.

Cell-Cell Junction

Tight Junctions are the closely associated areas of two cells whose membranes join together forming a virtually impermeable barrier to fluid. A desmosome is a cell structure specialized for cell-to-cell adhesion. A type of junction complex, they are localized spot-like adhesions randomly arranged on the lateral sides of plasma membranes. Desmosomes are molecular complexes of cell adhesion proteins and linking proteins that attach the cell surface adhesion proteins to intracellular keratin cytoskeletal filaments.

The cell adhesion proteins of the desmosome, desmoglein (DSG) and desmocollin (DSC), are members of the cadherin family of cell adhesion molecules. They are biomarker of skin tight junctions. In particular, DSG1 is a biomarker for cell binding, the higher the expression, the better skin cell-cell junction and the better skin barrier function will be. DSC3 is a protein in humans that is encoded by the DSC3 gene, the higher the expression, the better skin cell-cell junction and the better skin barrier function will be.

In the present method, keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (DSC1 and DSG3) expression level using a One-step TaqMan® RT-PCR kit (Life Technologies).

Test Results

Aqueous solutions of acetyl hexapeptide-46 and acetyl hexapeptide-38 were prepared by dilution to achieve the concentrations shown in Table 1. Acetyl hexapeptide-46 was obtained from Lipotec under the tradename Delisens.™ Delisens™ is a proprietary blend of butylene glycol, water, citric acid and acetyl hexapeptide-46, containing 0.025 wt. % acetyl hexapeptide-46. Acetyl hexapeptide-38 was obtained from Lipotec under the tradename Adifyline™ Adifyline™ is a proprietary blend of butylene glycol, water and acetyl hexapeptide-38, containing 0.05 wt. % acetyl hexapeptide-38.

The concentration of acetyl hexapeptide shown in the following table represents the concentration of the active ingredient. Thus, for example, in preparing Example 1A, 2 grams of Adifyline® was mixed with 98 g deionized water to prepare a solution that was 2 wt % Adifyline and 10 parts per million by weight (ppm) acetyl hexapeptide-38, based upon the total weight of the solution.

The samples were tested for IL-8 secretion as described in the test method above. That is, for Control A, human dermal keratinocytes were left untreated. For Control B, IL-8 a surfactant mixture that was a combination of sodium laureth sulfate and polyquaternium-10. For all other samples, the human dermal keratinocytes were co-treated with the surfactant mixture and a composition containing the ingredient of interest. Decreased 11-8 expression reflects the ingredient's anti-irritation activity. The results are summarized in Table 2 and shown graphically in FIG. 1. As can be seen in FIG. 1, both acetyl hexapeptide-38 and acetyl hexapeptide-46 reduce the IL-8 secretion, when compared to Control B.

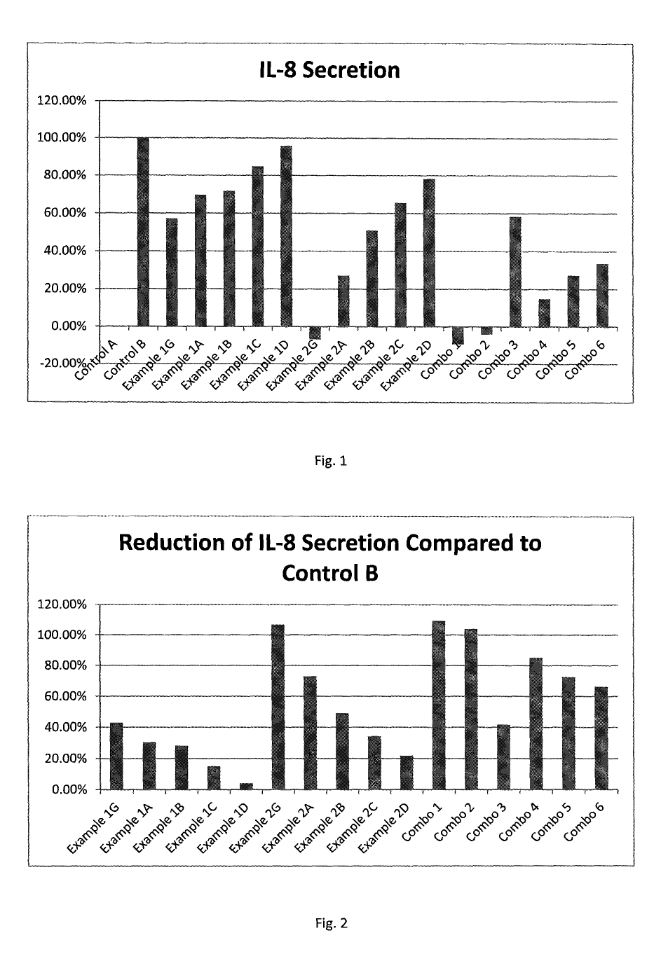

The amount of the reduction in IL-8 secretion for the test samples (subtracting from 100% for Control B) is summarized in Table 2 and shown graphically in FIG. 2. It can be seen that the combination of acetyl hexapeptide-46 and acetyl hexapeptide-38 produce an enhanced reduction in IL-8 secretion, particularly for the higher concentrations.

TABLE 1

| | | Acetyl | | Acetyl |
|---|---|---|---|---|
| | Adifyline | Hexapeptide- | Delisens | Hexapeptide- |
| Example | Wt. % | 38 ppm | Wt. % | 46 ppm |
| 1G | 3 | 15 | — | — |
| 1A | 2 | 10 | — | — |
| 1B | 1 | 5 | — | — |
| 1C | 0.5 | 2.5 | — | — |
| 1D | 0.2 | 1 | — | — |
| 2G | — | — | 3 | 7.5 |
| 2A | — | — | 2 | 5 |
| 2B | — | — | 1 | 2.5 |
| 2C | — | — | 0.5 | 1.25 |
| 2D | — | — | 0.2 | 0.5 |
| Combo 1 | 3 | 15 | 3 | 7.5 |
| Combo 2 | 2 | 10 | 2 | 5 |
| Combo 3 | 1 | 5 | 1 | 2.5 |
| Combo 4 | 1 | 5 | 2 | 5 |
| Combo 5 | 0.5 | 2.5 | 2 | 5 |
| Combo 6 | 0.2 | 1 | 2 | 5 |

TABLE 2

| | | Reduction of | |
|---|---|---|---|
| | | IL-8 Secretion | |
| | | Compared to | Standard |
| Example | IL-8 secreted | Control B | Deviation |
| Control A | 0.00% | — | 2.00% |
| Control B | 100% | — | 16.70% |
| 1G | 57.20% | 42.80% | 4.40% |
| 1A | 69.60% | 30.40% | 18.60% |
| 1B | 71.70% | 28.30% | 6.60% |
| 1C | 84.90% | 15.10% | 9.60% |
| 1D | 95.80% | 4.20% | 11.90% |
| 2G | −6.90% | 106.90% | 1.10% |
| 2A | 27.10% | 72.90% | 2.70% |
| 2B | 50.90% | 49.10% | 4.60% |
| 2C | 65.60% | 34.40% | 5.00% |
| 2D | 78.20% | 21.80% | 4.00% |
| Combo 1 | −9.40% | 109.40% | 1.10% |
| Combo 2 | −4.10% | 104.10% | 1.10% |
| Combo 3 | 58.30% | 41.70% | 6.10% |
| Combo 4 | 14.90% | 85.10% | 0.40% |
| Combo 5 | 27.40% | 72.60% | 3.60% |
| Combo 6 | 33.70% | 66.30% | 2.60% |

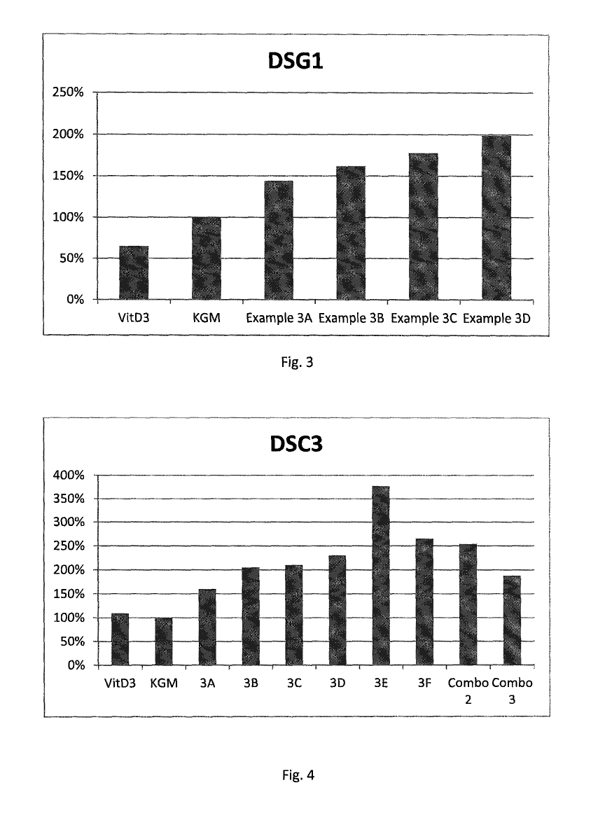

Samples containing various amounts of acetyl hexapeptide-38 and/or acetyl hexapeptide-46 were tested for cell-cell junction, as described above. Solution of Vitamin D3 and keratinocyte growth medium (KGM) were used for comparison. The results are summarized in Table 3 below, and graphically represented in FIGS. 3 and 4. It can be seen that compositions containing acetyl hexapeptide-38 and/or acetyl hexapeptide-46 enhanced cell-cell junction when compared to Vitamin D3 and KGM.

TABLE 3

| | | Conc. | DSG1 | DSC3 |
|---|---|---|---|---|
| Example | Composition | (ppm) | % | % |
| | VitD3 | | 65 | 109 |
| | KGM | | 100 | 100 |
| 3A | Acetyl hexapeptide-38 | 15 | — | 160 |
| 3B | Acetyl hexapeptide-38 | 7.5 | 144 | 205 |
| 3C | Acetyl hexapeptide-38 | 5 | 178 | 210 |
| 3D | Acetyl hexapeptide-46 | 7.5 | — | 230 |
| 3E | Acetyl hexapeptide-46 | 5 | 162 | 377 |
| 3F | Acetyl hexapeptide-46 | 2.5 | 199 | 265 |
| Combo 2 | Acetyl hexapeptide-38 + -46 | 5 + 2.5 | — | 254 |
| Combo 3 | Acetyl hexapeptide-38 + -46 | 10 + 5 | — | 188 |

Samples were prepared and tested as described above, except that pentapeptides were employed instead of hexapeptides. The concentrations of the samples tested are shown below, as well as the test results. The concentration refers to the parts per million by weight of active in the sample tested.

TABLE 4

| | | | Reduction of | |
|---|---|---|---|---|
| | Concentration | | IL-8 Secretion | |
| | Pentapeptide | | Compared to | |
| Example | (ppm) | IL-8 secreted | Control B | MTT |
| Control A | — | 0.00% | — | 100 |
| Control B | — | 100% | — | 57 |
| 4A | 1 | 67 | 33 | 80 |
| 4B | 5 | 59 | 41 | 81 |
| 4C | 10 | 58 | 42 | 82 |
| 4D | 15 | 64 | 36 | 78 |
| 4E | 20 | 85 | 15 | 75 |
| 5A | 0.2 | 47 | 53 | 81 |
| 5B | 0 | 10 | 90 | 54 |
| 5C | 2 | −3 | 100+ | 37 |
| 5D | 2.5 | −6 | 100+ | 32 |
| 5E | 3 | 0 | 100 | 21 |
| 5F | 3.5 | 21 | 79 | 12 |
| 6A | 5 | 6 | 94 | 97 |
| 6B | 15 | 5 | 95 | 94 |
| 6C | 25 | 9 | 91 | 65 |
| 6D | 35 | 6 | 94 | 44 |
| 6E | 40 | 3 | 97 | 32 |

It can be seen that compositions containing the tested pentapeptides produce an enhanced reduction in IL-8 secretion, and that they produce an enhanced cell-cell junction when compared to Vitamin D3 and KGM.

Various modifications and alterations that do not depart from the scope and spirit of this invention will become apparent to those skilled in the art. This invention is not to be duly limited to the illustrative embodiments set forth herein.

We claim:

1. A composition for enhancing skin barrier function, the composition comprising two or more oligopeptides.

2. The composition of claim 1, wherein the oligopeptides each include 12 amino acid moieties or less.

3. The composition of claim 1, wherein the oligopeptides each include 10 amino acid moieties or less.

4. The composition of claim 1, wherein the oligopeptides each include 8 amino acid moieties or less.

5. The composition of claim 1, wherein the oligopeptides each include at least 5 amino acid moieties.

6. The composition of claim 1, wherein the two or more oligopeptides are selected from the group consisting of acetyl hexapeptides.

7. The composition of claim 6, wherein the two or more oligopeptides include acetyl hexapeptide-38 and acetyl hexapeptide-46.

8. The composition of claim 1, wherein the two or more oligopeptides include at least one pentapeptide.

9. The composition of claim 8, wherein the pentapeptide is selected from the group consisting of acetyl pentapeptide-1, pamitoyl pentapeptide-3, and myristoyl pentapeptide-17.

10. The composition of claim 8, wherein the pentapeptide is selected from the group consisting of pamitoyl pentapeptide-3, and myristoyl pentapeptide-17.

11. A method for the cleansing and/or sanitizing skin, the method comprising the steps of:
combining a skin cleansing or skin sanitizing composition with one or more oligopeptides, wherein the oligopeptide reduces the irritancy potential of the composition; and
contacting the skin with the reduced-irritancy composition in a manner sufficient to cleanse and/or sanitize the skin.

12. A method for improving the barrier function of skin, the method comprising the steps of:
contacting the skin with an effective amount of a composition comprising two or more oligopeptides.

13. The method of claim 12, wherein the two or more oligopeptides are selected from the group consisting of acetyl hexapeptides.

14. The method of claim 13, wherein the two or more oligopeptides include acetyl hexapeptide-38 and acetyl hexapeptide-46.

15. The method of claim 12, wherein the two or more oligopeptides include at least one pentapeptide.

16. The method of claim 15, wherein the pentapeptide is selected from the group consisting of acetyl pentapeptide-1, pamitoyl pentapeptide-3, and myristoyl pentapeptide-17.
